(19) United States
(12) Patent Application Publication
(10) Pub. No.: US 2025/0082849 A1
(43) Pub. Date: Mar. 13, 2025

(54) MODULATING INSULIN UNDER CONDITIONS OF GASTROPARESIS

(71) Applicant: INSULET CORPORATION, Acton, MA (US)

(72) Inventor: Rangarajan NARAYANASWAMI, Weston, MA (US)

(21) Appl. No.: 18/827,167

(22) Filed: Sep. 6, 2024

Related U.S. Application Data
(60) Provisional application No. 63/582,042, filed on Sep. 12, 2023.

(51) Int. Cl. A61M 5/172

(52) U.S. Cl. CPC A61M 5/1723; A61M 2205/502

(57) ABSTRACT

In an aspect, an apparatus for generating a temporal element of a medication is presented. The apparatus includes a display device and a processor in electronic communication with the display device. The apparatus includes a memory communicatively connected to the processor. The memory includes instructions configuring the processor to generate a user interface through the display device and receive user input from a user through the user interface. The processor is configured to receive biological data from a user through a biological sensor in communication with the processor. The biological data includes a gastric metric. The processor is configured to calculate a temporal element of a delivery of an amount of medication to the user based on the biological data and the user input. The processor is configured to communicate the temporal element of the delivery of the amount of medication to a wearable medication delivery device.

### CROSS-REFERENCE TO RELATED APPLICATIONS

This application claims priority to and the benefit of U.S. Provisional Application No. 63/582,042, filed Sep. 12, 2023, the entirety of which is incorporated herein by reference.

### FIELD

The present subject matter generally relates to gastroparesis. In particular, the present subject matter relates to an apparatus for modulating insulin under conditions of gastroparesis.

### BACKGROUND

Gastroparesis is a complication that affects patients with Type 1 or Type 2 diabetes because of delayed gastric emptying of food. For patients with diabetes and gastroparesis, insulin dosing remains challenging. The timing of bolus doses for meals and the varying gastric emptying speed can increase the risk of early post prandial hypoglycemia and delayed hyperglycemia and even ketoacidosis. The incidence of gastroparesis increases with duration of the disease.

### SUMMARY OF THE DISCLOSURE

In an aspect, an apparatus for modifying a medication bolus delivery event is presented. The apparatus includes a display device and a processor in electronic communication with the display device. The apparatus includes a memory communicatively connected to the processor. The memory includes instructions configuring the processor to generate a user interface through the display device and receive user input from a user through the user interface. The processor is configured to receive biological data from a user through a biological sensor in communication with the processor. The biological data includes a gastric metric. The processor is configured to receive instructions for a medication bolus delivery event in which a bolus of medication is to be delivered to the user. The processor is configured to calculate a temporal element of the medication bolus delivery event based on the biological data and the user input. The processor is configured to generate instructions for a modified bolus delivery event to a wearable medication delivery device. The processor is configured to communicate the modified bolus delivery event to a wearable medication delivery device.

In another aspect, a system for modifying a medication bolus delivery event is presented. The system includes a computing device configured to generate a user interface through a display device and receive user input from a user through the user interface. The computing device is configured to receive biological data from the user through a biological sensor in communication with the computing device. The biological data includes a gastric metric. The computing device is configured to receive instructions for a medication bolus delivery event in which a bolus of medication is to be delivered to the user. The computing device is configured to calculate a temporal element of the medication bolus delivery event based on the biological data and the user input. The computing device is configured to generate instructions for a modified bolus delivery event based on the temporal element. The system includes a wearable medication delivery device in communication with the computing device. The wearable medication delivery device is configured to receive the temporal element of the delivery of an amount of medication to the user and deliver the amount of medication to the user through an injection mechanism.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

FIG. 1 illustrates a block diagram of an exemplary embodiment of an apparatus for generating a temporal element of a medication;

FIG. 2 illustrates a flow diagram of a process for enabling a gastroparesis mode of a wearable medication delivery device;

FIGS. 3A-C illustrate exemplary embodiments of graphical user interfaces according to the present disclosure;

FIGS. 4A-B depict exemplary embodiments of graphical user interfaces according to the present disclosure;

FIG. 5 illustrates a flow diagram of a feedback loop for a gastroparesis mode of a wearable medication delivery device; and

FIG. 6 illustrates an exemplary embodiment of a block diagram of a wearable injection device.

### DETAILED DESCRIPTION

In the following description, for the purposes of explanation, numerous specific details are set forth in order to provide a thorough understanding of the present invention. It will be apparent, however, that the present invention may be practiced without these specific details. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims.

At a high level, aspects of the present disclosure relate to a gastroparesis mode of a wearable medication delivery device. Aspects of the present disclosure can be used to generate temporal elements to modify bolus deliveries. In some embodiments, aspects of the present disclosure can be used to generate a graphical user interface that a user may interact with to identify symptoms of gastroparesis. An apparatus may detect gastric metrics of a user and automatically initiate a gastroparesis mode to modify bolus deliveries based on the gastric metrics of the user.

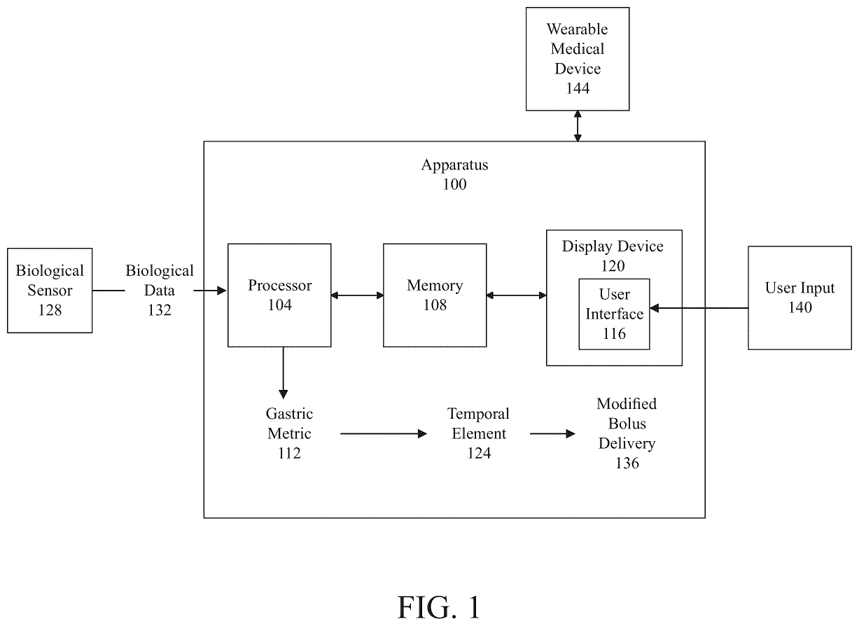

Referring now to FIG. 1A, apparatus 100 for glycemic control is presented. Apparatus 100 may include processor 104 and/or memory 108. As used in this disclosure, “communicatively connected” means connected by way of a connection, an attachment, or a linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Still referring to FIG. 1, the processor 104 may include a computing device. The processor 104 may include any computing device as described in this disclosure, including without limitation a microcontroller, microprocessor, digital signal processor (DSP) and/or system on a chip (SoC) as described in this disclosure. The processor 104 may include, be included in, and/or communicate with a mobile device such as a mobile telephone or smartphone. The processor 104 may include a single computing device operating independently, or may include two or more computing device operating in concert, in parallel, sequentially or the like; two or more computing devices may be included together in a single computing device or in two or more computing devices. The processor 104 may interface or communicate with one or more additional devices as described below in further detail via a network interface device (not shown). Network interface device may be utilized for connecting the processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. The processor 104 may include but is not limited to, for example, a computing device or cluster of computing devices in a first location and a second computing device or cluster of computing devices in a second location. The processor 104 may include one or more computing devices dedicated to data storage, security, distribution of traffic for load balancing, and the like. The processor 104 may distribute one or more computing tasks as described below across a plurality of computing devices of computing device, which may operate in parallel, in series, redundantly, or in any other manner used for distribution of tasks or memory between computing devices. Apparatus 100 may be implemented using a “shared nothing” architecture in which data is cached at the worker, in an embodiment, this may enable scalability of the processor 104 and/or another computing device.

With continued reference to FIG. 1, the processor 104, and/or any other computing device as described throughout this disclosure, may be designed and/or configured to perform any method, method step, or sequence of method steps in any embodiment described in this disclosure, in any order and with any degree of repetition. For instance, the processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Apparatus 100 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, in some embodiments, the apparatus 100 may be configured to receive biological data 132 from biological sensor 128. The apparatus 100 and/or the processor 104 may be in electronic communication with the biological sensor 128. “Electronic communication” as used in this disclosure is a form of connection between two objects where data is transferred. Electronic communication between the biological sensor 128 and the apparatus 100 may include, but is not limited to, wired, wireless, and/or other connections. A “biological sensor” as used in this disclosure is a device that detects biological data. The biological sensor 128 may include, without limitation, heart rate monitors, blood pressure sensors, blood oxygen sensors, thermometers, blood glucose monitors, continuous blood glucose monitors (CGM), and the like. The biological sensor 128 may detect and/or generate biological data 132. “Biological data” as used in this disclosure is information pertaining to a user's biology. The biological data 132 may include, without limitation, temperatures, blood pressures, heart rates, heart rhythms, blood oxygen levels, blood glucose levels, and the like. The apparatus 100 may receive the biological data 132 at the processor 104. In some embodiments, the apparatus 100 may receive the biological data 132 from a Bluetooth, Wi-Fi, or other connection from one or more external computing devices. The processor 104 may store the biological data 132 in the memory 108. The processor 104 may utilize the biological data 132 that may be stored in the memory 108 to determine one or more trends, patterns, and the like.

In FIG. 1, biological data 132 and/or user input 140 may include a gastric metric 112. A “gastric metric” as used in this disclosure is information pertaining to a digestive system of an individual. The gastric metric 112 may include, without limitation, a rate of digestion of an individual, a relative delay in digestion of an individual, and the like. For instance, the gastric metric 112 may show a delayed gastric emptying of an individual. The gastric metric 112 may be obtained through the user input 140 and/or from the biological data 132. In some embodiments the biological data 132 may show a delayed increase in blood glucose after a user has indicated through the user input 140 that they are eating. The processor 104 may determine the gastric metric 112 to be a delayed digestion of a user of about 20 minutes, without limitation. In some embodiments, the gastric metric 112 may include a relative metabolism of a user. For instance, the processor 104 may determine, based on the biological data 132 and/or the user input 140, that a user has a higher metabolism and may digest foods more quickly, that the user has a slower metabolism and may digest foods more slowly, or that the user has an average metabolism. The gastric metric 112 may include relative metabolisms of a user and may utilize relative metabolisms of the user to generate the modified bolus delivery event 136. In some embodiments, the processor 104 may calculate the modified bolus delivery event 136 based on the metabolism of the user as a permanent setting and may adjust the modified bolus delivery event 136 and/or a detected metabolism through readings of the biological data 132.

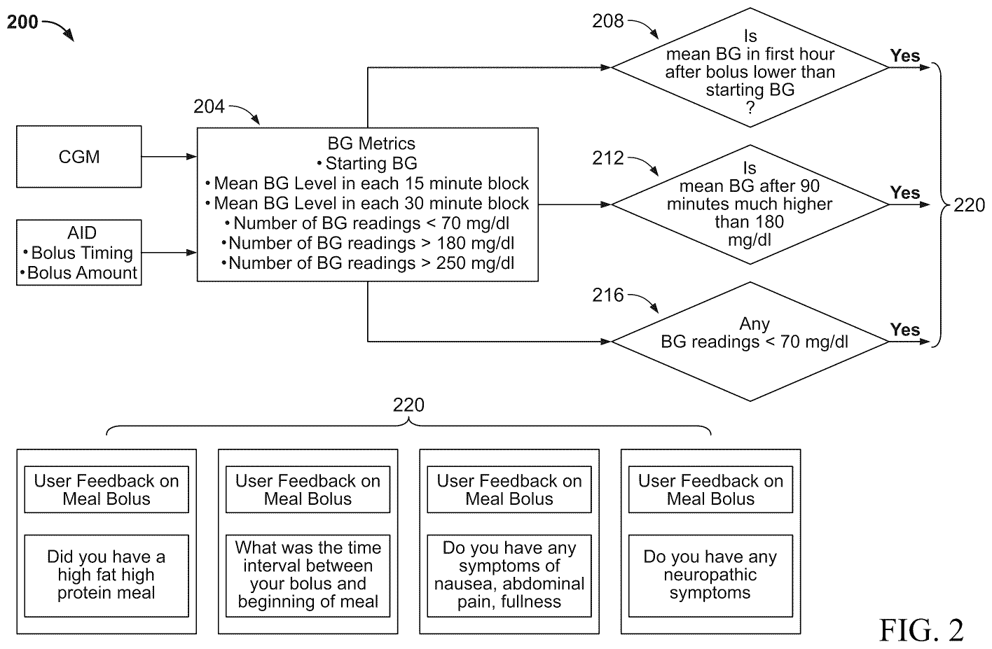

In some embodiments, the processor may be programmed to cause the user interface 116 to display a list of symptoms related to gastroparesis, such as, but not limited to, nausea, abdominal pain, fullness, and the like. Symptoms displayed through user interface 116 may include neuropathic symptoms. The user interface 116 may display a list of one or more gastric symptoms to a user. A “gastric symptom” as used in this disclosure is an indication of an affliction relating to gastroparesis. Gastric symptoms may include, but are not limited to, nausea, abdominal pain, fullness, and/or other symptoms. In some embodiments, gastric symptoms may include neuropathic symptoms. “Neuropathic symptoms” as used in this disclosure are neurological signs of gastroparesis. Neuropathic symptoms may include, but are not limited to, numbness, loss of sensation, pain in extremities, and the like. The user interface may display a list of gastric and/or neuropathic symptoms as described below with reference to FIG. 2.

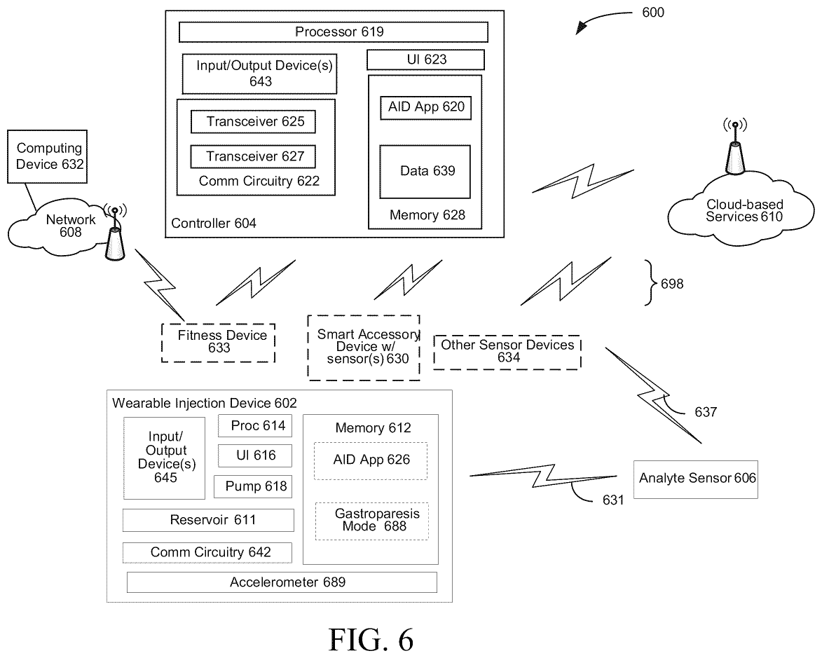

The processor 104 may be configured to calculate the gastric metric 112 and/or the temporal element 124 based on the user input 140 received through the user interface 116 displaying a list of gastric symptoms. For instance, the processor 104 may compare one or more selected gastric symptoms presented through the user interface 116 to one or more thresholds for diagnosing gastroparesis. A threshold for diagnosing gastroparesis may include a positive indication of one or more gastric symptoms. As a non-limiting example, a user may select gastric symptoms of nausea and fullness, to which the processor 104 may determine the user is experiencing gastroparesis and determine the temporal element 124 to include a time delay of digestion for the user of about 20 minutes. Determining and/or calculating the temporal element 124 may include comparing previous gastric metrics 112 with previous temporal elements 124, calculating average periods of time of delayed digestion of one or more users, generating trends of gastric metrics 112 to blood glucose levels, and the like. In some embodiments, gastric symptoms may include neuropathic symptoms such as, without limitation, pain in feet, loss of balance, loss of sensation, and the like. The user interface 116 may prompt a user to enter and/or select one or more gastroparesis symptoms, neuropathic symptoms, and the like. In some embodiments, the processor 104 may receive the gastric metric 112 based on the user input 140 received through the user interface 116. In some embodiments, the processor 104 may obtain and/or derive the gastric metric 112 through the biological data 132. The processor 104 may be in communication with a wearable medication delivery device, such as the wearable injection device described below in FIG. 6.

The processor 104 may monitor blood glucose levels of a user through the biological data 132 and/or monitor insulin levels, delivery times of medication, and the like, through data communicated with a wearable medication delivery device. The processor 104 may determine a misalignment of a delivery of a medication with a sensed physiological condition, such as a misalignment of insulin delivery with a blood glucose level of a user. A misalignment may include a delivery of insulin before a rise in blood glucose levels of a user.

For instance, blood glucose levels may be low initially followed by elevated blood glucose levels, such as in a hyperglycemic state. The processor 104 may determine that a user is experiencing delayed gastric emptying and may generate the gastric metric 112 to include an approximate or exact delay in gastric emptying of the user, such as for example a delay of about 20 minutes. In some embodiments, the processor 104 may determine the glycemic metric 112 includes a temporal anomaly and generate a prompt for a user through user interface 116, such as with one or more questions relating to the user's digestive actions. A “temporal anomaly” as used in this disclosure is an irregularity in blood glucose levels of a user within a timeframe. A temporal anomaly may include, without limitation, lower than expected blood glucose levels, higher than expected blood glucose levels, unchanged blood glucose levels, and the like. The processor 104 may determine a temporal anomaly by comparing an expected blood glucose level to an actual blood glucose level. Above a certain threshold in a difference between an expected blood glucose level and an actual blood glucose level may prompt the processor 104 to flag the blood glucose level reading. A threshold in a difference between an expected blood glucose level and an actual blood glucose level may include, without limitation, 5%, 10% and the like. In some embodiments, the processor 104 may automatically generate the modified bolus delivery event 136 based on a detected temporal anomaly in blood glucose levels of a user. The processor 104 may alert a user, such as through sounds or visual indicators communicated through the display device 120, of a detected temporal anomaly.

Still referring to FIG. 1, the processor 104 may determine temporal element 124 based on the gastric metric 112, the user input 140, the biological data 132, and/or other forms of data. A “temporal element” as used in this disclosure is a time variable relating to a delivery of a medication. The temporal element 124 may include, but is not limited to, a time period of seconds, minutes, hours, and the like. The temporal element 124 may include a delay in delivering a bolus of medication. In some embodiments, the temporal element 124 may include a preemptive delivery of at least a portion of a bolus of medication.

The processor 104 may determine a gastric phase of a user based on the biological data 132, the gastric metric 112, the temporal element 124, and/or the user input 140. A “gastric phase” as used in this disclosure is a step of digestion of an individual. A gastric phase may include, but is not limited to, ingestion, mechanical processing, chemical and/or physical digestion, secretion, absorption, and/or excretion. In some embodiments, the processor 104 may determine a gastric phase of a user through the biological data 132 and/or the user input 140. For instance, the user input 140 may include a user's selection of a button displayed through the user interface 116 indicating the user has started eating a meal. The processor 104 may correlate the indication of the user starting to eat a meal with an ingestion phase. In some embodiments, the processor 104 may determine patterns of gastric phases of a user. For instance, the processor 104 may determine that a user ingests food over a time span of about 10 minutes every morning at 9 AM. The processor 104 may further determine, through blood glucose levels of the biological data 132, that the user has a delayed chemical and/or physical digestion stage of about 25 minutes after an ingestion stage. The processor 104 may correlate food and/or meal types to delayed gastric phases of a user, such as a digestion and/or ingestion stage. For instance, the processor 104 may correlate foods with complex carbohydrates with slower and/or delayed digestion phase times. In some embodiments, the processor 104 may display one or more graphs, trendlines, and the like, of correlations between food and/or meal types and digestive phases of a user, such as through the display device 120.

Referring still to FIG. 1, the processor 104 may generate and/or receive instructions for a modified bolus delivery event 136 of a medication bolus delivery event. A medication bolus delivery event may include a period of time over which an upfront amount of medication is delivered to a user. A “bolus delivery” as used in this disclosure is an administering of medication to a user. The modified bolus delivery event 136 may include a delivery of medication, such as insulin. The modified bolus delivery event 128 may be generated based on the temporal element 124 and/or the gastric metric 112. For instance, in an illustrative embodiment, the gastric metric 112 may show delayed gastric emptying of a user and the temporal element 124 may show a calculated delay for administering a medication of about 12 minutes. A calculated delay may be calculated through comparison of gastric metrics 112 to blood glucose levels, determining one or more trends based on historical biological data 132, and the like. Accordingly, the modified bolus delivery event 136 may include a delay of about 12 minutes. In some embodiments, the modified bolus delivery event 136 may include one or more waveforms of medication delivery over a period of time. For instance, and without limitation, the modified bolus delivery event 136 may include a sine wave, cosine wave, square wave, and/or other waveform, each of which may include one or more frequencies, amplitudes, peak to peak lengths, and the like. A square wave of the modified bolus delivery event 136 may include periods of no medication delivery followed by periods of short but high medication delivery. The modified bolus delivery event 136 may include a square wave that may align with periods of delayed digestion of a user. As a non-limiting example, a user may ingest food and the biological data 132 may show no significant change in blood glucose readings of the user. The processor 104 may determine the gastric metric 112 to include delayed digestion and the temporal element 124 to include a time period of about 35 minutes. The processor 104 may generate the modified bolus delivery event 136 based on the biological data 132, the gastric metric 112, and/or the temporal element 124. The modified bolus delivery event 136 may include a square wave of medication delivery over a time period, which may align with the time period of the temporal element 124, such as about 35 minutes. In other words, the modified bolus delivery event 136 may include an initial time delay of about 35 minutes rather than an immediate upfront delivery as soon as the user has begun eating.

The processor 104 may generate the modified bolus delivery event 136 for a temporary period of time, such as, but not limited to, minutes, hours, and the like. The processor 104 may determine that a user may be experiencing normal blood glucose readings through the biological data 132 and cease modifications to the modified bolus delivery event 136. Ceasing modifications to the modified bolus delivery event 136 may result in the modified bolus delivery event 136 resembling an undelayed bolus delivery. An unmodified bolus delivery may include a bolus delivery that is delivered upfront as soon as a user starts eating or as soon as the user requests a meal bolus. The processor 104 may correlate food types, days, and the like, to temporary periods of time of generating the modified bolus delivery event 136. For instance and without limitation, the processor 104 may determine that on Monday mornings, a user typically needs the modified bolus delivery event 136 for about 2.5 hours, at which point the user may experience normal blood glucose readings and/or gastric metrics 112.

Apparatus 100 may include and/or be connected to display device 120. A “display device” as used in this disclosure is an object that displays information through a screen. The display device 120 of apparatus 100 may, in some embodiments, include a liquid crystal display (LCD), organic light emitting diode display (OLED), and/or other displays. The display device 120 may include a touchscreen. The touchscreen may be responsive to resistive touch, capacitive touch, and/or other forms of touch input. “Touch input” as used in this disclosure is a form of data communication through a touch sensitive device. Touch input may include, but is not limited to, user input such as tapping, double tapping, triple tapping, long presses, swipes, and the like. In some embodiments, touch input may include input received from one or more styluses. A “stylus” as used in this disclosure is an object configured to interact with a touchscreen. A stylus may include, but is not limited to, a capacitive stylus, resistive stylus, and the like. A user may interact with the touchscreen of the display 120 through a use of one or more styluses.

Apparatus 100 may display user interface 116 through display device 120. User interface 116 may include a graphical user interface (GUI), without limitation. A “graphical user interface” as used in this disclosure is a form of communication with a computing device through one or more pictorial icons. The user interface 116 may include one or more event handlers. An “event handler” as used in this disclosure is a callback routine that operates asynchronously once an event takes place. An event handler of the user interface 116 may be linked with one or more graphical icons of a GUI of the user interface 116. For instance, and without limitation, an event handler may be programmed to generate a pop-up window of a settings menu upon a click of a settings icon displayed through the user interface 116. In some embodiments, the user interface 116 may include a mobile application, web portal, and/or other form of interface. The user interface 116 may display biological data 132, such as, without limitation, blood glucose levels.

Still referring to FIG. 1, in some embodiments, the apparatus 100 may be, or preferably include, a wearable medication delivery device 144. A “wearable medication delivery device” as used in this disclosure is a computing device affixed to a user that is configured to monitor and/or affect a user's health. Wearable medication delivery device 144 may comprise a drug delivery system as described below with reference to FIG. 6, such as an insulin pump. In some embodiments, the apparatus may comprise, without limitation, smart watches, heart rate monitors, and the like. In some embodiments, the apparatus 100 may communicate data with a wearable medication delivery device, such as through a wired, wireless, or other connection. The processor 104 may communicate the modified bolus delivery event 136 with a wearable medication delivery device, such as the wearable medication delivery device described below with reference to FIG. 6.

FIG. 2 illustrates an exemplary embodiment of a flowchart of an AID process 200 for incorporating delays in gastric emptying. At step 204 blood glucose metrics are measured. Blood glucose metrics may include, without limitation, levels or concentrations of blood glucose of an individual, average blood glucose levels or concentrations over a period of time, and the like. Blood glucose metrics may include a starting blood glucose. A starting blood glucose may include an initial blood glucose level of a user before the user eats a meal. In alternative embodiments, a starting blood glucose may include a blood glucose level of a user when the user requests a meal bolus; or alternatively a blood glucose level of a user immediately after the user eats a meal. Blood glucose metrics may be measured in intervals, such as, but not limited to, intervals of 1 minute, 5 minutes, 15 minutes, 30 minutes, 1 hour, and the like. The AID process 200 may record a number of blood glucose readings above or below certain thresholds. For instance, a number of blood glucose readings below 70 mg/dL, above a pre-established setpoint such as 100 mg/dL or 110 mg/dL, above 180 mg/dL, or above 250 mg/dL may be counted, without limitation.

At step 208, a mean blood glucose of a user in a first hour after receiving a bolus of medication is compared to a starting blood glucose level. If the mean blood glucose after the first hour is not lower than the starting blood glucose, the process 200 continues to step 212. In some embodiments, if the mean blood glucose after the first hour is lower than the starting blood glucose, the process 200 may continue to step 212.

At step 212, a mean blood glucose after 90 minutes is compared to a blood glucose value of 180 mg/dl. If the mean blood glucose is not higher than 180 mg/dl, process 200 goes onto step 216. In some embodiments, if the mean blood glucose is higher than 180 mg/dl, the process 200 may continue to step 216.

At step 216, a count of blood glucose readings under 70 mg/dl is measured. If no blood glucose readings are under 70 mg/dl, process 200 may stop or end. In some embodiments, if no blood glucose readings are under 70 g/dl, process 200 may loop back to step 204.

Referring back to steps 208-216, if any of the comparisons are true, process 200 continues to step 220. At step 220, user prompts are generated. In some embodiments, a single user prompt may be generated. In some embodiments, two or more user prompts may be generated. User prompts may include text displayed on one or more user interfaces. User prompts may include prompts such as, but not limited to, “Did you have a high fat high protein meal?”, “What was the time interval between your bolus and beginning of meal?”, “Do you have any symptoms of nausea, abdominal pain, or fullness?”, “Do you have any neuropathic symptoms such as loss of sensation?”. A user may answer one or more prompts, which may trigger a gastroparesis mode of a wearable medication delivery device. In some embodiments, a certain combination of positive answers to one or more user prompts may trigger a gastroparesis mode of a wearable medication delivery device. For instance, a user may answer no to the first user prompt, enter a time interval of 1 hour at the second user prompt, and answer yes to the third and fourth user prompts, which may cause an initiation of a gastroparesis mode. As another non-limiting example, a user may answer no to the first user prompt, give a time interval of 30 minutes to the second user prompt, answer yes to the third user prompt, and answer no to the fourth user prompt. In this instance, a gastroparesis mode may also be initiated.

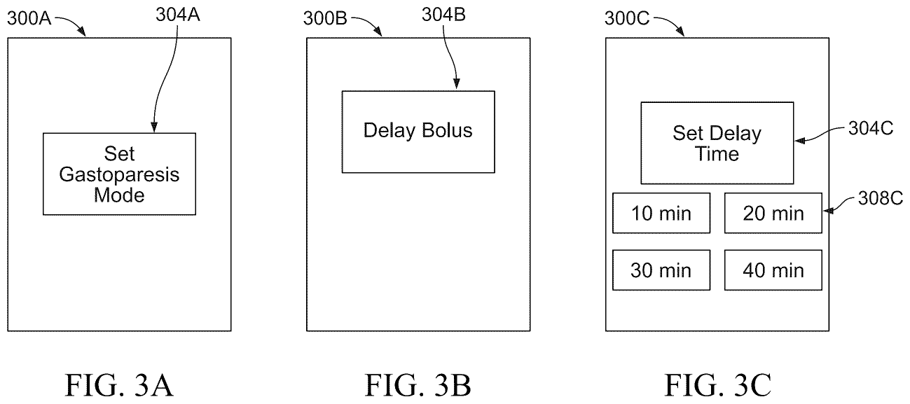

Referring now to FIG. 3A, a graphical user interface 300A is presented. GUI 300A may include one or more graphics, icons, and the like. GUI 300A may be configured to receive user input, such as, but not limited to, touch input, mouse input, keyboard input, and the like. GUI 300A may be presented on one or more display devices such as laptops, smartphones, tablets, and the like. GUI 300A may include set mode button 304A. The set mode button 304A may be a rectangular, square, or other shape. In some embodiments, the set mode button 304A may be an orange rectangle. The set mode button 304A may display text, such as “Set Gastroparesis Mode”. The set mode button 304A may correspond to a gastroparesis mode of a wearable medication delivery device, such as described above with reference to FIG. 1.

Referring now to FIG. 3B, graphical user interface 300B is presented. GUI 300B may be the same as GUI 300A as described above. In some embodiments, GUI 300B may include bolus delay button 304B. The bolus delay button 304B may be rectangular, square, and/or another shape. In some embodiments, the bolus delay button 304B may be an orange rectangular button. In some embodiments, the bolus delay button 304B may include text, such as “Delay Bolus”, without limitation. The bolus delay button 304B may correspond to a delay in a delivery of a bolus of medication. In some embodiments, a user may interact with the delay bolus button 304B which may cause GUI 300B to animate to GUI 300C as described below.

Referring now to FIG. 3C, GUI 300C is presented. GUI 300C may be the same as that of GUI 300A as described above with reference to FIG. 3A. GUI 300C may include a time delay button 304C. The time delay button 304C may be rectangular, square, or another shape. In some embodiments, the time delay button 304C may be an orange rectangle. The time delay button 304C may be responsive to user input. For instance, a user may tap the time delay button 304C which may generate time interval windows 308B. In some embodiments, a user may interact with the time interval windows 308B to select a specific delay of time and then interact with the time delay button 304C to save the selection of the time interval window 308C. In some embodiments, a user may interact with GUI 300A which may prompt GUI 300B and then GUI 300C sequentially in response to the user interaction. In some embodiments, only GUI 300A may be displayed and the gastroparesis mode may be automated to calculate a bolus delay.

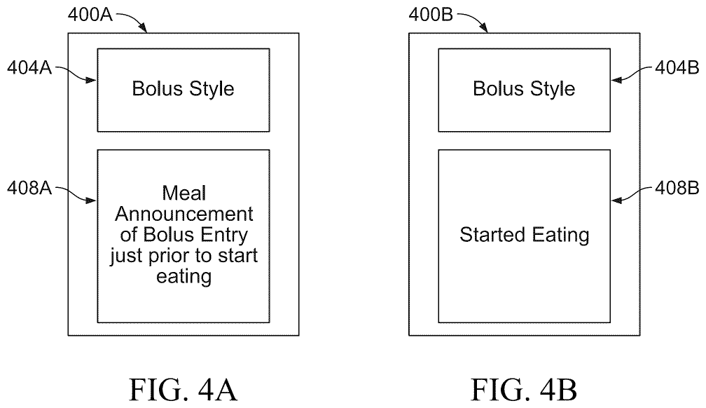

Referring now to FIG. 4A, GUI400A is presented. The GUI 400A may include one or more icons, graphics, and the like. In some embodiments, the GUI 400A may be the same as that of the GUI 300A as described above with reference to FIG. 3A. The GUI 400A may be displayed on, but not limited to, smartphones, tablets, laptops, and the like. In some embodiments the GUI 400A may include and/or display header icon 404A. The header icon 404A may include an icon in a shape of a rectangle, square, circle, and the like. The header icon 404A may be shaped as a rectangle having an orange color. In some embodiments, the header icon 404A may include one or more text boxes. A text box of the header icon 404A may display text, such as, but not limited to, characters, symbols, words, and the like. In an embodiment, the header icon 404A may display text reading “Bolus Style”. The headier icon 404A may be representative of one or more actions a user may select through the GUI 400A. For instance, the header icon 404A may display text saying “Bolus Style” which may be representative of a form of an amount of medication a wearable medication delivery device may use.

The GUI 400A may display and/or include timing button 408A. The timing button 408A may include one or more icons, pictures, and the like. In some embodiments, the timing button 408A may be a rectangular box having a green color. In other embodiments, the timing button 408A may be a square, circle, and the like which may have a red, blue, yellow, and/or other color, without limitation. The timing button 408A may display text through one or more text boxes. For instance, the timing button 408A may display text stating “Meal Announcement” or “Bolus entry Prior to Eating”. The timing button 408A may be positioned below the header icon 404A. A user may interact with the timing button 408A with touch input, keyboard input, mouse input, and/or other forms of user input, without limitation.

Referring now to FIG. 4B, a GUI 400B is presented. The GUI 400B may be the same as that of the GUI 400A as described above with reference to FIG. 4A. The GUI 400B may display header icon 404B and timing icon 408B, which may be the same as that of the header icon 404A and the timing icon 408B as described above with reference to FIG. 4A. In some embodiments, the timing icon 408B may display text reading “Started Eating”, or other text indications of an initiation of consumption of a user. A user may interact with the timing icon 408B through one or more of touch input, mouse input, keyboard input, and the like, without limitation.

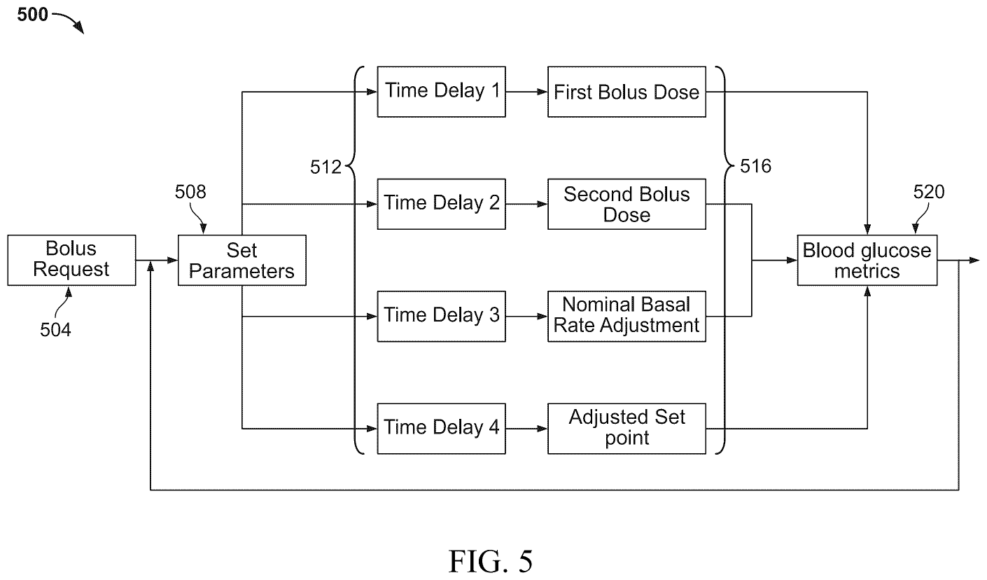

Referring now to FIG. 5, a flowchart of an AID process 500 for timing boluses is illustrated. At step 504, an initial bolus request is generated. A bolus request may include an amount of medication requested by a user and/or computing device. For instance, the bolus request may include an initiation of an amount of insulin to be delivered through a wearable medication delivery device. A user may manually generate the bolus request and/or a wearable medication delivery device may automatically initiation a bolus request. At step 508, parameters of the bolus request of step 504 are set. Parameters may include, but are not limited to, quantity of medicine, delivery rate of medicine, frequency of medicine delivery, and the like. Step 508 may include setting one or more time delays 512. A time delay may include a period of time such as, but not limited to, seconds, minutes, hours, and the like. At step 508, one or more time delays 512 may be generated. In some embodiments, a single time delay 512 may be generated. In other embodiments, two or more time delays 512 may be generated. Each time delay of the time delays 512 may correspond to one or more medication delivery parameters 516, such as, but not limited to, bolus quantity, bolus timing, nominal basal rates, and/or set points of blood glucose of a user. For instance, and without limitation, a first time delay of the time delays 512 may correspond to a first bolus dose of the medication delivery parameters 516, a second time delay of the time delays 512 may correspond to a second bolus dose of the medication delivery parameters 516, a third time delay of the time delays 512 may correspond to a basal rate of the medication delivery parameters 516, and a fourth time delay of the time delays 512 may correspond to an adjust blood glucose set point of the medication delivery parameters 516. As a non-limiting example, a first time delay of the time delays 512 may include a 20 minute delay of a delivery of medication and a first bolus does of the medication delivery parameters 516 may include an adjusted initial bolus dose of about 5% of a total daily insulin value (TDI), a second time delay may include 60 minutes and a second bolus may include 10% of a TDI value, a third time delay may include 15 minutes and a nominal basal rate may be increased 20% from a reference value, and a fourth time delay may include 90 minutes and an adjusted set point may include a reduction to 90 mg/dl.

With continued reference to FIG. 5, at step, 520, blood glucose metrics are received. Blood glucose metrics may be received from a sensing device such as, but not limited to, a blood glucose monitor, continuous blood glucose monitor, and the like. The blood glucose metrics may be received at a computing device, such as a smartphone, wearable medication delivery device, and the like. The blood glucose metrics may include, without limitation, average blood glucose readings, current blood glucose readings, changes in blood glucose readings, and the like. For instance, and without limitation, the blood glucose readings may have a current blood glucose reading of 110 mg/dl. The blood glucose metrics measured at step 520 may be input into step 508 in a feedback loop. In other words, the blood glucose metrics read at step 520 may influence the time delays 512 and/or the medication delivery parameters 516.

A bolus delivery may be calculated by Equation 1:

$$Safe Required IOB = ( \frac{Current BG - ( Setpoint + Elevation )}{Correction Factor} ) * TDI$$

Where BG is a blood glucose, setpoint is a target blood glucose value, elevation is an added increase in a blood glucose target setpoint, TDI is a total daily insulin, and a correction factor is a value out of a TDI. The correction factor may be calculated using the “1800 rule” which depicts how much a blood glucose level of a user may drop per unit of insulin delivered. For instance, if a user takes 30 units of insulin a day, the calculation may be 1800/30 which equals 60. This would indicate the user's blood sugar would drop by 60 mg/dl per unit of insulin delivered. A meal dose may be calculated using Equation 2:

$$meal dose = x \% TDI + safe Required IOB - Current Total IOB$$

Where meal dose is an amount of medication, x is a percent value of a TDI value, the TDI is a total daily insulin, the correction factor is 1800/TDI, the safe required IOB is a minimum safe amount of insulin on board and the current total IOB is the current total insulin on board. As a non-limiting example, x can be 3%, 8%, or another percentage of a TDI value. In some embodiments, a bolus calculator may calculate a bolus using Equation 3:

$$Correction = ( \frac{Current BG - ( Setpoint )}{CF} )$$

Where the correction is a set blood glucose value given by a difference between a current blood glucose level and a target setpoint level. The difference is divided by a correction factor. In some embodiments, a meal dose may be calculated using Equation 4:

$$meal dose = \frac{grams of carbs}{Insulin to Carbs Ratio} + Correction - Current Total IOB$$

Where the meal dose is an amount of insulin delivered for a given meal. A computing device, such as a wearable medication delivery device, may utilize one or more of Equations 1, 2, 3, and/or 4 to output a bolus of medication. For instance, at step 520, blood glucose metrics may be measured which may be input into any of the above equations which may adjust and/or correct a bolus delivery.

While the examples are described with primarily with reference to insulin for ease of discussion, the disclosed systems, devices and techniques are more broadly applicable to a medication of which there are a variety. Examples of medications may include any drug in liquid form capable of being administered by a drug delivery device via a subcutaneous cannula, including, for example, insulin, glucagon-like peptide-1 (GLP-1), pramlintide, glucagon, co-formulations of two or more of GLP-1, and pramlintide; as well as pain relief drugs, such as opioids or narcotics (e.g., morphine, or the like), methadone, arthritis drugs, hormones, such as estrogen and testosterone, Alzheimer drugs, blood pressure medicines, chemotherapy drugs, fertility drugs, or the like.

FIG. 6 illustrates an exemplary embodiment of a drug delivery system Referring now to FIG. 6, a block diagram of a drug delivery system 600 is illustrated. In some examples, the drug delivery system 600 is suitable for delivering insulin to a user in accordance with the disclosed embodiments. The drug delivery system 600 may include a wearable medication delivery device 602, a controller 604 and an analyte sensor 606. In addition, the drug delivery system may interact with a computing device 632 via a network 608 as well as interact with cloud-based services 610 via a wireless connection, such as a cellular data network or the like.

The wearable medication delivery device 602 may be a wearable device that is worn on the body of the user. The wearable medication delivery device 602 may be directly coupled to a user (e.g., directly attached to the skin of the user via an adhesive, or the like, at various locations on the user's body, such as thigh, abdomen, or upper arm). In an example, a surface of the wearable medication delivery device 602 may include an adhesive to facilitate attachment to the skin of the user.

In an example, the wearable medication delivery device 602 may include a processor 614. The processor 614 may be implemented in hardware, software, or any combination thereof. The processor 614 may, for example, be a microprocessor, a logic circuit, a field programmable gate array (FPGA), an application specific integrated circuit (ASIC) or a microprocessor coupled to a memory. The processor 614 may maintain a date and time as well as be operable to perform other functions (e.g., calculations or the like). The processor 614 may be operable to execute a control application 626 and/or a voice control application stored in the memory 612 that enables the processor 614 to direct operation of the wearable medication delivery device 602. The control application 626 may control insulin delivery to the user utilizing an AID algorithm. The memory 612 may store settings 624 that may include AID application settings for a user, such as specific factor settings, subjective insulin need parameter settings, and AID algorithm settings, such as maximum insulin delivery, insulin sensitivity settings, total daily insulin (TDI) settings and the like. The memory 612 may also store other data 629, related to control and operation (e.g., status information of a power supply (not shown), reservoir level, event history, and operating history), and the like.

Still referring to FIG. 6, the input/output device(s)645 may one or more of a microphone, a speaker, a vibration device, a display, a push button, a touchscreen display, a tactile input surface, or the like. The input/output device(s) 645 may be coupled to the processor 614 and may include circuitry operable to generate signals based on received inputs and provide the generated signals to the processor 614. In addition, the input/output device(s) 645 may be operable to receive signals from the processor 614 and, based on the received signals, generate outputs via a respective output device.

Still referring to FIG. 6, the wearable medication delivery device 602 may include a reservoir 611. The reservoir 611 may be operable to store drugs, medications or therapeutic agents suitable for automated delivery, such as insulin, morphine, methadone, hormones, glucagon, glucagon-like peptide, blood pressure medicines, chemotherapy drugs, combinations of drugs, such as insulin and glucagon-like peptide, or the like. A fluid path to the user may be provided via tubing and a needle/cannula (not shown). The fluid path may, for example, include tubing coupling the wearable medication delivery device 602 to the user (e.g., via tubing coupling a needle or cannula to the reservoir 611). The wearable medication delivery device 602 may be operable based on control signals from the processor 614 to expel the drugs, medications or therapeutic agents, such as insulin, from the reservoir 611 to deliver doses of the drugs, medications or therapeutic agents, such as the insulin, to the user via the fluid path. For example, the processor 614 by sending control signals to the pump 618 may be operable to cause insulin to be expelled from the reservoir 611.

Still referring to FIG. 6, there may be one or more communication links 698 with one or more devices physically separated from the wearable drug delivery device 602 including, for example, a controller 604 of the user and/or a caregiver of the user and/or a sensor 606. The analyte sensor 606 may communicate with the wearable medication delivery device 602 via a wireless communication link 631 and/or may communicate with the controller 604 via a wireless communication link 637. The communication links 631, 637, and 698 may include wired or wireless communication paths operating according to any known communications protocol or standard, such as Bluetooth, Wi-Fi, a near-field communication standard, a cellular standard, or any other wireless protocol.

Still referring to FIG. 6, the wearable medication delivery device 602 may also include a user interface (UI) 616, such as an integrated display device for displaying information to the user, and in some embodiments, receiving information from the user. For example, the user interface 616 may include a touchscreen and/or one or more input devices, such as buttons, knob or a keyboard that enable a user to provide an input.

Still referring to FIG. 6, in addition, the processor 614 may be operable to receive data or information from the analyte sensor 606 as well as other devices, such as smart accessory device 630, fitness device 633 or another wearable device 634 (e.g., a blood oxygen sensor or the like), that may be operable to communicate with the wearable drug delivery device 602. For example, fitness device 633 may include a heart rate sensor and be operable to provide heart rate information or the like.

Still referring to FIG. 6, the wearable medication delivery device 602 may interface with a network 608. The network 608 may include a local area network (LAN), a wide area network (WAN) or a combination therein and operable to be coupled wirelessly to the wearable medication delivery device 602, the controller, and devices 630, 633, and 634. A computing device 632 may be interfaced with the network 608, and the computing device may communicate with the wearable medication delivery device 602. The computing device 632 may be a healthcare provider device, a guardian's computing device, or the like through which a user's controller 604 may interact to obtain information, store settings, and the like. The AID application 620 may be operable to execute an AID algorithm and present a graphical user interface on the computing device 632 enabling the input and presentation of information related to the AID algorithm. The computing device 632 may be usable by a healthcare provider, a guardian of the user of the wearable medication delivery device 602, or another user.

Still referring to FIG. 6, the wearable medication delivery device 602 may include an analyte sensor 606 for detecting the levels of one or more analytes of a user, such as blood glucose levels, ketone levels, other analytes relevant to a diabetic treatment program, or the like. The analyte level values detected may be used as physiological condition data and be sent to the controller 604 and/or the wearable medication delivery device 602. The sensor 606 may be coupled to the user by, for example, adhesive or the like and may provide information or data on one or more medical conditions and/or physical attributes of the user. The sensor 606 may be a continuous glucose monitor (CGM), ketone sensor, or another type of device or sensor that provides blood glucose measurements that is operable to provide blood glucose concentration measurements. The sensor 606 may be physically separate from the wearable medication delivery device 602 or may be an integrated component thereof. The analyte sensor 606 may provide the processor 614 and/or processor 619 with physiological condition data indicative of measured or detected blood glucose levels of the user. The information or data provided by the sensor 606 may be used to modify an insulin delivery schedule and thereby cause the adjustment of drug delivery operations of the wearable medication delivery device 602.

Still referring to FIG. 6, the analyte sensor 606 may be operable to collect physiological condition data, such as the blood glucose measurement values and a timestamp, ketone levels, heart rate, blood oxygen levels and the like that may be shared with the wearable medication delivery device 602, the controller 604 or both. For example, the communication circuitry 642 of the wearable medication delivery device 602 may be operable to communicate with the analyte sensor 606 and the controller 604 as well as the devices 630, 633 and 634. The communication circuitry 642 may be operable to communicate via Bluetooth®, Wi-Fi, a near-field communication standard, a cellular standard, or any other wireless protocol.

Still referring to FIG. 6, in the depicted example, the controller 604 may include a processor 619 and a memory 628. The controller 604 may be a special purpose device, such as a dedicated personal diabetes manager (PDM) device. The controller 604 may be a programmed general-purpose device that is a portable electronic device, such as any portable electronic device, smartphone, smartwatch, fitness device, tablet or the like including, for example, a dedicated processor, such as processor, a micro-processor or the like. The controller 604 may be used to program or adjust operation of the wearable medication delivery device 602 and/or the sensor 606. The processor 619 may execute processes to manage a user's blood glucose levels and that control the delivery of the drug or a therapeutic agent (e.g., a liquid drug or the like as mentioned above) to the user. The processor 619 may also be operable to execute programming code stored in the memory 628. For example, the memory 628 may be operable to store an AID application 620 for execution by the processor 619. The AID application 620 may be responsible for controlling the wearable medication delivery device 602, including the automatic delivery of insulin based on recommendations and instructions from the AID algorithm, such as those recommendations and instructions described herein.

Still referring to FIG. 6, the memory 628 may store one or more applications, such as an AID application 620, a voice control application, gastroparesis mode 688, and other data 639 which may be the same as, or substantially the same as those described above with reference to the wearable medication delivery device 602. In addition, the settings 621 may store information, such as drug delivery history, blood glucose measurement values over a period of time, total daily insulin values, and the like. The memory 628 may be further operable to store other data 639, such as blood glucose history, medication delivery history, HbA1C history, programming code and libraries, and the like. In addition, the memory may store settings 621, which may include AID settings and parameters, insulin treatment program history (such as insulin delivery history, blood glucose measurement value history) and the like. Other parameters such as insulin-on-board (IOB) and insulin-to-carbohydrate ratio (ICR) may be retrieved from prior settings and insulin history stored in memory. For example, the control application 620 may be operable to store the AID algorithm settings, such as blood glucose target set points, insulin delivery constraints, basal delivery rate, insulin delivery history, wearable drug delivery device status, and the like. The memory 628 may also be operable to store data such as a food database for carbohydrate (or macronutrient) information of food components (e.g., grilled cheese sandwich, coffee, hamburger, brand name cereals, or the like). The memory 628 may be accessible to the AID application 620 and a voice control application.

Still referring to FIG. 6, the input/output device(s) 643 of the controller 604 may one or more of a microphone, a speaker, a vibration device, a display, a push button, a tactile input surface, or the like. The input/output device(s) 643 may be coupled to the processor 619 and may include circuitry operable to generate signals based on received inputs and provide the generated signals to the processor 619. In addition, the input/output device(s) 643 may be operable to receive signals from the processor 619 and, based on the received signals, generate outputs via one or more respective output devices, such as a speaker, a vibration device, or a display.

Still referring to FIG. 6, the controller 604 may include a user interface (UI) 623 for communicating visually with the user. The user interface 623 may include a display, such as a touchscreen, for displaying information provided by the AID application 620 or a voice control application. The touchscreen may also be used to receive input when it is a touch screen. The user interface 623 may also include input elements, such as a keyboard, button, knob or the like. In an operational example, the user interface 623 may include a touchscreen display controllable by the processor 619 and be operable to present the graphical user interface, and in response to a received input (audio or tactile), the touchscreen display is operable present a graphical user interface related to the received input.

Still referring to FIG. 6, the controller 604 may interface via a wireless communication link of the wireless communication links 698 with a network, such as a LAN or WAN or combination of such networks that provides one or more servers or cloud-based services 610 via communication circuitry 622. The communication circuitry 622, which may include transceivers 627 and 625, may be coupled to the processor 619. The communication circuitry 622 may be operable to transmit communication signals (e.g., command and control signals) to and receive communication signals (e.g., via transceivers 627 or 625) from the wearable medication delivery device 602 and the analyte sensor 606. In an example, the communication circuitry 622 may include a first transceiver, such as 625, that may be a Bluetooth transceiver, which is operable to communicate with the communication circuitry 622 of the wearable medication delivery device 602, and a second transceiver, such as 627, that may be a cellular transceiver, a Bluetooth® transceiver, a near-field communication transceiver, or a Wi-Fi transceiver operable to communicate via the network 608 with computing device 632 or with cloud-based services 610. While two transceivers 625 and 627 are shown, it is envisioned that the controller 604 may be equipped more or less transceivers, such as cellular transceiver, a Bluetooth transceiver, a near-field communication transceiver, or a Wi-Fi transceiver.

Still referring to FIG. 6, the cloud-based services 610 may be operable to store user history information, such as blood glucose measurement values over a set period of time (e.g., days, months, years), a drug delivery history that includes insulin delivery amounts (both basal and bolus dosages) and insulin delivery times, types of insulin delivered, indicated meal times, blood glucose measurement value trends or excursions or other user-related diabetes treatment information, specific factor settings including default settings, present settings and past settings, or the like.

Still referring to FIG. 6, other devices, like smart accessory device 630 (e.g., a smartwatch or the like), fitness device 633 and other wearable device 634 may be part of the drug delivery system 600. These devices may communicate with the wearable medication delivery device 602 to receive information and/or issue commands to the wearable medication delivery device 602. These devices 630, 633 and 634 may execute computer programming instructions to perform some of the control functions otherwise performed by processor 614 or processor 619. These devices 630, 633 and 634 may include user interfaces, such as touchscreen displays for displaying information such as current blood glucose level, insulin on board, insulin deliver history, or other parameters or treatment-related information and/or receiving inputs. The display may, for example, be operable to present a graphical user interface for providing input, such as request a change in basal insulin dosage or delivery of a bolus of insulin. Devices 630, 633 and 634 may also have wireless communication connections with the sensor 606 to directly receive blood glucose level data as well as other data, such as user history data maintained by the controller 604 and/or the wearable medication delivery device 602.

Still referring to FIG. 6, the user interface 623 may be a touchscreen display controlled by the processor 619, and the user interface 623 is operable to present a graphical user interface that offers an input of a subjective insulin need parameter usable by the AID application 620. The processor 619 may cause a graphical user interface to be presented on the user interface 623. Different examples of the graphical user interface may be shown with respect to other examples. The AID application 620 may generate instructions for the pump 618 to deliver basal insulin to the user or the like.

Still referring to FIG. 6, the processor 619 is also operable to collect physiological condition data related to the user from sensors, such as the analyte sensor 606 or heart rate data, for example, from the fitness device 633 or the smart accessory device 630. In an example, the processor 619 executing the AID algorithm may determine a dosage of insulin to be delivered based on the collected physiological condition of the user and a specific factor determined based on the subjective insulin need parameter. The processor 619 may output a control signal via one of the transceivers 625 or 627 to the wearable drug delivery device 602. The outputted signal may cause the processor 614 to deliver command signals to the pump 618 to deliver an amount of related to the determined dosage of insulin in the reservoir 611 to the user based on an output of the AID algorithm. The processor 619 may also be operable to perform calculations regarding settings of the AID algorithm as discussed as herein. Modifications to the AID algorithm settings provided via the voice control application 621, such as by the examples described herein, may be stored in the memory 628.

Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts requesting the input of information such as weight or age. Similarly, a dosage amount of insulin may be received by the AP application or algorithm from a user via a user interface. Other open-loop actions may also be implemented by adjusting user settings or the like in an AP application or algorithm.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. An apparatus for modifying a medication bolus delivery event, comprising:
a display device;
a processor in electronic communication with the display device; and
a memory communicatively connected to the processor, the memory containing instructions configuring the processor to:
generate a user interface through the display device and receive user input from a user through the user interface;
receive biological data from the user through a biological sensor in communication with the processor, wherein the biological data includes a gastric metric;
receive instructions for a medication bolus delivery event in which a bolus of medication is to be delivered to the user;
calculate a temporal element of the medication bolus delivery event based on the biological data and the user input;
generate instructions for a modified bolus delivery event based on the temporal element; and
communicate the modified bolus delivery event to a wearable medication delivery device.

2. The apparatus of claim 1, wherein the processor is further configured to present a list of gastric symptoms through the user interface to a user.

3. The apparatus of claim 2, wherein the processor is further configured to receive user input relating to the list of gastric symptoms; and
calculate the temporal element based on the user input.

4. The apparatus of claim 1, wherein the processor is further configured to determine a gastric phase of the user and generate the instructions for the modified bolus delivery event based on the gastric phase of the user.

5. The apparatus of claim 1, wherein the processor is further configured to alert the user of a detected temporal anomaly.

6. The apparatus of claim 5, wherein the processor is further configured to automatically generate the instructions for the modified bolus delivery event based on the detected temporal anomaly.

7. The apparatus of claim 1, wherein the instructions for the modified bolus delivery event comprise a waveform of the amount of medication to be delivered in the modified bolus delivery event.

8. The apparatus of claim 1, wherein the processor is further configured to present a time delay button through the user interface; and
generate the instructions for the modified bolus delivery event based on user input received through the time delay button of the user interface.

9. The apparatus of claim 1, wherein the processor is further configured to determine a metabolism of the user and generate the instructions for the modified bolus delivery event based on the metabolism.

10. The apparatus of claim 1, wherein the processor is further configured to instruct the medication delivery device to deliver the modified bolus delivery event to the user.

11. A system for modifying a medication bolus delivery event, comprising:
a computing device configured to:
generate a user interface through the display device and receive user input from a user through the user interface;
receive biological data from the user through a biological sensor in communication with the computing device, wherein the biological data includes a gastric metric;
receive instructions for a medication bolus delivery event in which a bolus of medication is to be delivered to the user;
calculate a temporal element of the medication bolus delivery event based on the biological data and the user input;
generate instructions for a modified bolus delivery event on the temporal element; and
a wearable medication delivery device in communication with the computing device, wherein the wearable medication delivery device is configured to:
receive the instructions for the modified bolus delivery from the computing device; and
deliver the amount of medication of the modified bolus delivery to the user through an injection mechanism.

12. The system of claim 11, wherein the computing device is further configured to present a list of gastric symptoms through the user interface to a user.

13. The system of claim 12, wherein the computing device is further configured to
receive user input relating to the list of gastric symptoms; and
calculate the temporal element based on the user input.

14. The system of claim 11, wherein the computing device is further configured to determine a gastric phase of the user and generate the instructions for the modified bolus delivery event based on the gastric phase of the user.

15. The system of claim 11, wherein the computing device is further configured to alert the user of a detected temporal anomaly.

16. The system of claim 15, wherein the computing device is further configured to automatically generate the instructions for the modified bolus delivery event based on the detected temporal anomaly.

17. The system of claim 11, wherein the instructions for the modified bolus delivery event comprise a waveform of the amount of medication to be delivered in the modified bolus delivery event.

18. The system of claim 11, wherein the computing device is further configured to present a time delay button through the user interface; and
generate the instructions for the modified bolus delivery event based on user input received through the time delay button of the user interface.

19. The system of claim 11, wherein the computing device is further configured to determine a metabolism of the user and generate the instructions for the modified bolus delivery event based on the metabolism.

20. The system of claim 11, wherein the computing device is further configured to instruct the medication delivery device to deliver the modified bolus delivery to the user.
